(19) United States
(12) Patent Application Publication
(10) Pub. No.: US 2008/0177480 A1
(43) Pub. Date: Jul. 24, 2008

(54) Procedure for the acquisition, treatment and transmission of data linked to human energy consumption during daily activity, and/or sporting practices and a device therefor

(76) Inventors: Stefano Infriccioli, Merano (IT); Amedeo Infriccioli, Acquavivra Picena (IT)

Correspondence Address: PYLE & PIONTEK;ATTN: THOMAS R. VIGIL, 221 N LASALLE STREET , ROOM 2036, ROOM 2036, CHICAGO, IL, 60601, US

(21) Appl. No.: 12/004,247

(22) Filed: Dec. 20, 2007

(30) Foreign Application Priority Data
Jun. 29, 2005 (IT) M02005A000166

(51) Int. Cl. A61B 5/11

(52) U.S. Cl. 702/19

(57) ABSTRACT

The present invention relates to a field of procedures for the acquisition, treatment and transmission data linked to human energy consumption. A first activation phase (1a) of a device containing at least one acceleration sensor element and at least one temperature element and a microcontroller element is followed at least by a phase (3) to check for an active connection with a connected remote device, followed by a calibration phase (5), followed by a phase (6) to detect motion, followed by a phase (6b) to detect movement of the user's foot, followed by a phase (9) to detect the type of movement and a phase (11) to calculate and memorize the velocity, the movement and the energy.

### TECHNICAL FIELD

The present invention relates to a procedure for the acquisition, treatment and transmission of data linked to human energy consumption during daily activity and/or sporting practices and a device therefor.

### BACKGROUND ART

It should be noted preliminarily that, simply speaking, the term ‘metabolism’ can be used to define that complex of chemical reactions that are performed in every living organism giving rise to a quantity of energy which is yielded externally to said organism in the form of thermal power and mechanical power, supplying aforesaid organism with the energy necessary to carry out various types of activities including therein physical activity. It is however known that the energy introduced into the body by means of the assumption of alimentary substances does not necessarily coincide with the quantity of energy dissipated during the various physical activities and in the event that the energy introduced exceeds that dissipated, there is an accumulation of energy inside said body with the results and difficulties known by all. It appears therefore obvious that the information relating to human energy consumption takes on considerable importance both in the event that said information, yielded in numerical form, is exploitable directly by the person concerned and also in the event that said information can be sent, exploiting the Internet, to a dietician who, in this way, appears to become a virtual figure so to speak.

There have been many attempts until now both to render information relating to human energy consumption available in numerical form and to render the figure of the dietician virtual.

Starting from the situation in which, practically, the only device available to measure the energy consumption was constituted of a set of scales and the only means available to transmit the data of interest was constituted of the filling in of certain forms on a web page, a considerably important step ahead was made with the invention of the so-called “pedometer”. This device, incorporating an acceleration sensor, is designed to count steps and, to do this successfully and considering that when a person walks, their pelvis has a rhythmic falling and rising movement, a pendulum algorithm is applied; the acceleration sensor with which the pedometer is fitted serves for “feeling” said movement and, consequently, calculating the steps effectuated; it is, therefore, an indirect evaluation method. The major drawback of the “pedometer” is represented by the difficulty in tracing from the number of steps effectuated back to the distance effectively travelled since said distance depends greatly on the walking “style”; it appears therefore comprehensible how this method can easily be deceived since the person who completes a certain course can do it, for example, jumping, running or spinning around.

From the explanation above, it appears obvious that the drawback of the “pedometer” could also be identified as the difficulty in tracing from the number of steps back to the energy consumed.

In fact, the realisation of the acceleration sensors, which can be on one, two or three axes, was important as it permitted the realisation of inertial navigation systems based on the mathematical link between the measurement of acceleration, velocity and movement; therefore it seems natural that there have been various attempts to utilise acceleration sensors to detect the movement of the human body.

Nevertheless, it should be noted that the application of said sensors, has always presented certain drawbacks, the most important of which being identifiable as the low accuracy of the data detectable.

In order to understand this drawback, a simple example follows, deduced from the “Analogue Device 2002” in the article “Using the ADXL202 in Pedometer and Personal Navigation Applications”, written by Harvey Weinberg.

By applying the formula:

$$Position ( t ) = Position ( 0 ) + \frac{Acceleration \times {t}^{*}}{2}$$

(where Position (0) refers to the starting position) in the event that one wishes to measure the movement of a person who is moving at a velocity of 5 Km/h for a duration of 5 minutes. The velocity of movement equals≈1.39 m/sec and the movement would be≈416 m; by applying said formula, the person's average acceleration would be:

$$Acceleration - 2 \times \frac{Position ( t ) - Positon ( 0 )}{{t}^{2}} \approx 0.00926 \underline{m} {sec}^{2}$$

which corresponds to an average acceleration value equalling≈0.944 mg.

It therefore appears obvious, first of all, that the value present is so low that it risks not even being detectable unless particularly sensitive acceleration sensors are utilised, i.e. those known as “low-g” sensors. Secondly, it should be noted that, since the temperature coefficient of said acceleration sensors is typically equal to 2 mg/° C., the useable signal is not even half the signal, due to the variation by a just one ° C. Furthermore, it should be noted that the error obtained is much greater in the event that the orientation of the acceleration sensor is not known with extreme accuracy, said fact constituting a further serious drawback of the devices realised until now. As far as the orientation of the acceleration sensor(s) is concerned, it should be noted that the output of said sensor(s) is generally either of the type whose voltage is proportional to the acceleration or of the type with a fixed frequency digital signal and with operational cycle proportional to the acceleration.

When acceleration occurs in the direction indicated, the result obtained is a positive number while if the acceleration occurs in the opposite direction to that indicated, a negative number is obtained. Naturally, at least two routes were taken in order to compensate for the scarce detection accuracy: a first solution was to zero out the acceleration sensor operations after brief intervals of time, together with an accurate knowledge of the orientation of said sensor obtained by means of the information supplied by an external device, for example a GPS, which periodically provides the new “Position (0)” value.

A second solution is to utilise an acceleration sensor designed to calculate the vertical movement of the pelvis of a human body which is executing a movement

It should also be noted that the methods proposed until now for the acquisition of data linked to human energy consumption present the drawback of being methods which utilise indirect methods to calculate the movement since what is, in actual fact, calculated is the number of steps taken over a certain period of time.

### DISCLOSURE OF INVENTION

A first aim of the present invention is constituted of the development of a method suitable to render available, in an accurate form, both directly to the person concerned and over the Internet, information concerning the energy consumption thereof over the course of both normal daily activity and sporting practices.

A further aim is to provide a method utilising signals supplied by a sensor located in proximity to an anatomical point positioned away from the user's torso.

In particular, the procedure for the acquisition, treatment and transmission, including therein via Internet, of data linked to human energy consumption during daily activity and/or sporting practices, of the type utilising data supplied by at least one acceleration sensor, in question in the present invention is characterised by the facts that it is composed of at least the following phases in succession:

activation of a device containing at least one acceleration sensor and at least one temperature sensor and a microcontroller;

check for an active connection with a connected remote device;

possible calibration of said device;

detection of the motion of at least one acceleration sensor element, correction of the samples acquired and memorisation of the samples and temperatures;

detection of the movement of the user's foot;

detection of the type of movement;

calculation and memorisation of the velocity and movement and corporeal energy.

This and further characteristics will better emerge in the detailed description that follows of a preferred embodiment of the invention, illustrated purely in the form of a non-limiting example in the plates enclosed, in which:

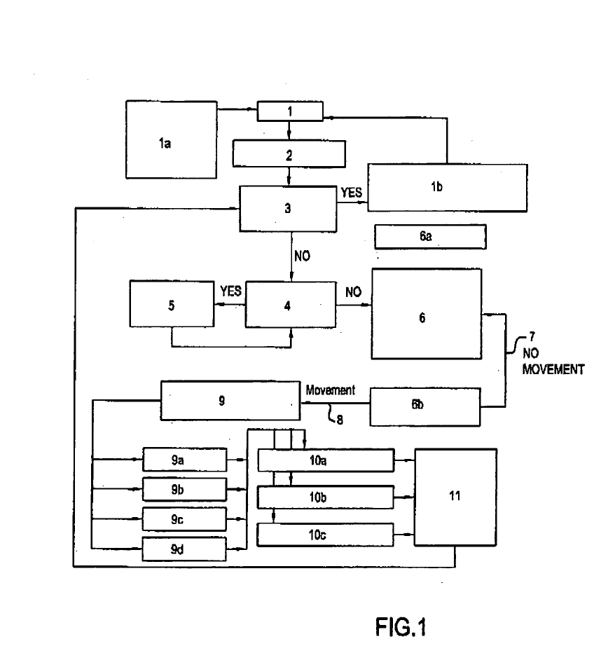

FIG. 1 illustrates a block diagram of the succession of the phases of the procedure;

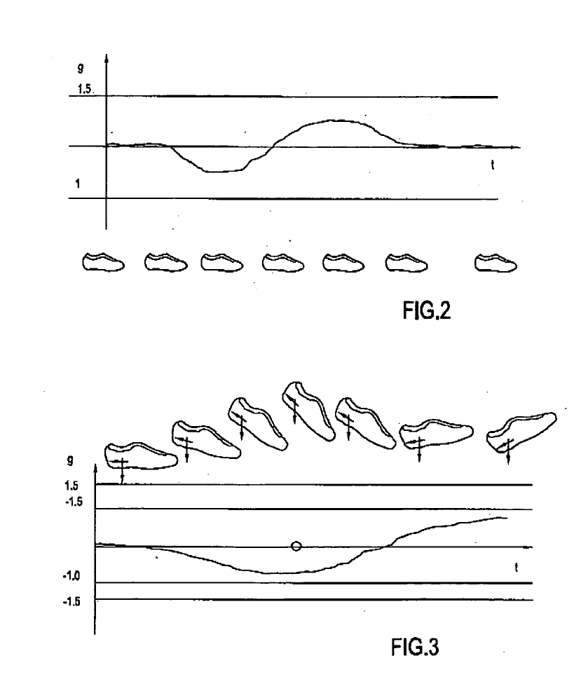

FIG. 2 illustrates, in diagram form, the course of the acceleration of gravity g as a function of the time t in the case of an acceleration sensor positioned inside the sole of a shoe;

FIG. 3 illustrates, in diagram form, the course of the acceleration of gravity g as a function of the time t outputted by the acceleration sensor where it is important to observe the interval +/−1.5 g above which the acceleration sensor saturates;

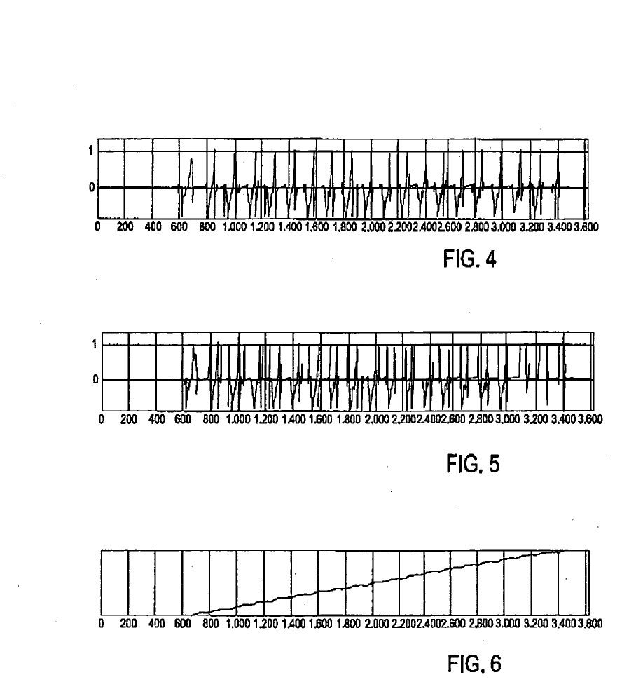

FIG. 4 illustrates, in a diagram g as function of t, the trend of the data acquisition from an acceleration sensor in the case of a person who has walked a distance of twenty-six meters;

FIG. 5 illustrates, in a diagram g as function of t, the contents of the previous figure combined with the density of the acceleration variation per unit of time;

FIG. 6 illustrates, in a diagram v as function of t, the trend of the velocity and the movement during the intervals of time in which the detection of movement is not zero;

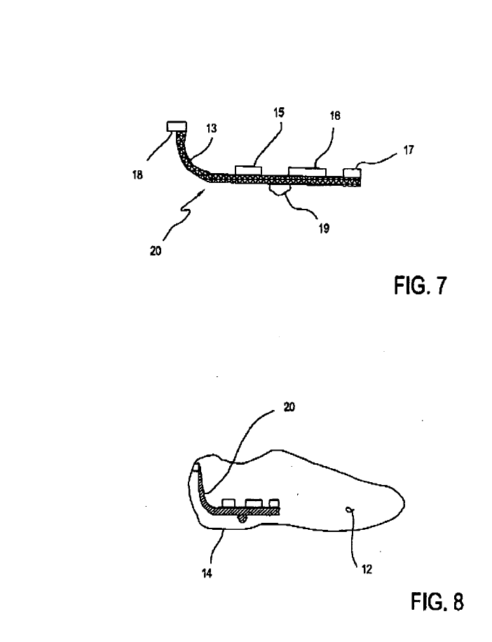

FIG. 7 illustrates a device for the actuation of a procedure for the acquisition, treatment and transmission, including therein via Internet, of data linked to human energy consumption during daily activity and/or sporting practices;

FIG. 8 shows the device inserted into a shoe. With reference to FIG. 1, number 1 refers to an initial phase of the data detection and 1a refers to a phase which actually starts the procedure, corresponding to the application of the shoe to the user's foot.

Subsequently to phase 1 there is a phase 2 corresponding to the initialisation of a microcontroller element 16 followed by a phase 3 indicating whether a remote access device is connected or not In the event that said connection is active, there follows a phase 1b which concerns either the method by which the parameters calculated are downloaded or the downloading of a new program. From phase 1b, the procedure moves back to the previous phase 1. In the event that the remote access device is not connected, the procedure moves on to phase 3 and to a phase 4 in which it is checked whether a calibration element is active or not; if the result is affirmative, there follows a phase 5 regarding the calibration procedure and then the procedure moves back to said phase 4 while, in the event of a negative result, the procedure enters a subsequent phase 6 regarding the procedure for the detection of motion of an acceleration sensor element, for correction of the data acquired and memorisation of said data and, finally, for memorisation of data regarding the corporeal temperature.

Aforesaid phase 6 can give rise to two subsequent phases: a stand-by phase 6a which leads back to phase 6 and a phase 1% in the event that a condition 7 corresponding to lack of movement occurs. In the event that a condition 8 occurs, i.e. the presence of movement, there follows a phase 9 regarding the detection of the typology of said movement. Concerning this, the method in question in the present invention takes into consideration four different typologies of movement, and more is precisely: a typology 9a, which is slow running, a typology 9b, which is fast running, a typology 9c defined as disturbed, referring—with this term—to all those movement typologies which are not indicated correctly by the algorithm of the microcontroller 16 such as, for example, a half step or a kick of a ball, and a typology 9d, which is jumping.

All four of said movement typologies can be divided into three kinds of activity: an activity 10a effectuated uphill, an activity 10b effectuated downhill and an activity 10c effectuated on level ground. From each of said three activities the procedure passes to a final phase 11 which is inherent to both the calculation and the memorisation of the velocity and the movement of the action effectuated by the user and also the calculation and memorisation of the energy consumed. At the end of phase 11, one moves back to phase 3.

Since, as it was previously highlighted, it is necessary to utilise acceleration sensor elements with a low g number, there is a risk that said sensor becomes saturated in the event that the foot accelerates too much, for example, when one runs. The said risk of saturation is also present, on the other hand, in the event of low velocity movements as a consequence of the fact that when one walks, the foot tends to rotate. Said saturation problem can be overcome as explained hereinafter and as illustrated by FIGS. 2 and 3.

Starting from the assumption that an acceleration sensor element 15 which saturates at±1.5 g is fixed to the lower portion of a shoe 12 so that when said shoe moves forwards horizontally, without any rotation, then slows down and stops, it generates the course illustrated by the graph in the diagram g as a function of t in FIG. 2. In the case in which the said acceleration sensor element is positioned and oriented as above mentioned and in the case in which the shoe rotates simply with no acceleration, there follows the course shown in FIG. 3, in which the position indicated by the dark dot represents the moment at which the horizontal acceleration begins to increase and from this instant onwards the gravitational acceleration starts to make the signal supplied by the acceleration sensor 15 increase, thereby preventing the saturation phenomenon being triggered.

The influence of the gravitational acceleration still generates a distortion of the signal supplied by said acceleration sensor element and this leads to the fact that, in order to have good accuracy in the measurement of the body's movement, it is necessary to calibrate the acceleration sensor and this occurs in phase 5 of the procedure described above. Said calibration phase begins exactly with phase la when the user puts on the shoe 12 and in doing so, presses an activation element 19 against the ground; after accomplishing this first operation, the user walks for a set distance stopping after this course and in this way a microcontroller element 16 exits the calibration phase 5 when it does not sense any type of movement for a fixed duration of time. Before ending said calibration phase, the microcontroller element updates and saves the parameters utilised by the algorithm designated to calculate the energy.

Concerning the recalibration operation, it should be noted that this is a good practice in order to obtain accurate measurements; in other words, and as is obvious, the better the data obtained is, the more updated the device inserted into the shoe is kept.

The contents of FIG. 4 constitute the course—shown in a gravitational acceleration diagram g as a function of time t expressed in seconds—of the acquisition of the data by the acceleration sensor 15 in the case of a person walking for 26 meters. Said microcontroller converts 140 samples per second.

Phase 6 analyses the motion of the acceleration sensor element 15 when the foot wearing the shoe fitted with said sensor is resting on the ground, said motion being due to the not perfect alignment of the foot with respect to the ground. In the event that said motion proves different from zero, during phase 6 there is a memorisation of this and there will be a subtraction thereof from the subsequent data; said motion is analysed step by step, bearing in mind that a slow variation, in the order of tenths of a second, means that the motion is due to the variation in the temperature while if the entity of the variation is more rapid, and also discontinuous, this means that the said motion is due to the inclination of the ground, which can be either ascending or descending; said detections are performed in the phases from 9 to 10c already described and illustrated in FIG. 1. It should be noted that the distinction between the variation in the motion due to the temperature to and the variation in the motion due to the inclination of the ground becomes commonplace in the event that there is a two-axis acceleration sensor available.

The course of the movement detection function analysing the density of the acceleration variation by unit of time is shown in FIG. 5 by means of the squared course superimposed on the graph shown in FIG. 4.

FIG. 6 displays the course of the velocity and the motion analysing the acceleration curve from FIG. 5 in the intervals of time in which the movement detecting function is not zero.

A first advantage of the procedure in question in the present invention is constituted of the development of a method suitable to render available, in an accurate form, information concerning a person's energy consumption over the course of both normal daily activity and sporting practices.

A further advantage is constituted of the rendering available of a method utilising signals supplied by a sensor located in proximity to an anatomical point positioned away from the user's torso.

A device for the acquisition and treatment of data linked to human s energy consumption during daily activity and/or sporting practices, of the type containing at least a printing circuit bearing, fixed integrally thereto, at least one acceleration sensor element, a voltage generation element, an activation element and at least one temperature sensor element, as per the present invention, characterised by the fact that said device is positioned inside a shoe and is fitted with at least one microcontroller element.

A first aim of the present invention is to realise a device utilising signals supplied by a sensor located inside a shoe in order to obtain signals which are decidedly superior to those supplied by sensors positioned in correspondence with other anatomical zones.

A farther aim is to realise a device which permits a direct movement calculation method.

A still further aim of the present invention consist of the rendering available, in an accurate form, both directly to the person concerned and over the Internet, information concerning the energy consumption thereof over the course of both normal daily activity and sporting practices.

With reference to FIG. 7 illustrating a device for the acquisition, treatment and transmission of data linked to human energy consumption during daily activity and/or sporting practices, number 12 refers to a shoe and 13 is a printed circuit positioned inside a sole 14. On the surface of the printed circuit 13 and fixed thereto are an acceleration sensor element 15, a microcontroller element 16, a voltage generation 17 and a temperature sensor element 18.

On the surface of said printed circuit is fixed an activation element 19, which, in tie case illustrated, is a pushbutton element. The assembly constituted of the printed circuit 13 and all the elements from 15 to 19 fixed thereto constitute a device 20.

When the user puts on the shoe 12 and presses said shoe against the ground, there occurs a pressing of the activation element 19 and, consequently, the device 20 begins to function. The data, generated by the acceleration sensor element 15 and the temperature sensor element 18, powered by the voltage generation element 17, are both converted and processed by the microcontroller element 16.

It should be noted that the anatomical position indicated improves the signal/noise relationship of the acceleration sensor element and due to the discontinuous nature of the movement of the feet, the thermal and mechanical drift can be eliminated.

Furthermore, the device is also fitted with an interface element whose utility could be appreciated in the event of a radio frequency implementation concerning the data transmission; this interface element has been grouped together, for simplicity, with the reference number 18 indicated in said FIG. 7.

A first advantage is that a device is realised utilising signals supplied by at least one acceleration sensor and at least one temperature sensor both located integrally to the foot in order to obtain signals which are decidedly superior to those supplied by sensors positioned in correspondence with other anatomical zones.

A further advantage is constituted of the realisation of a device which permits a direct movement calculation method.

A still further advantage consists in the rendering available, in an accurate form, both directly to the person concerned and over the Internet, information concerning the energy consumption thereof over the course of both normal daily activity and sporting practices.

I claim:

1. A procedure for the acquisition, treatment of data linked to human energy consumption during daily activity and/or sporting practices, of the type utilizing data supplied by at least one acceleration sensor, characterised by the fact that said procedure is composed of at least the following phases in succession:
activation (1a) of a device (20), containing at least one acceleration sensor element (15) and at least one temperature sensor element (18) and a microcontroller element (16);
check (3) for an active connection with a connected remote device;
eventual calibration (5) of said device;
detection (6) of the motion of at least one acceleration sensor (15), correction of the samples acquired and memorisation of the samples and the temperatures;
detection (6b) of the movement of the user's foot;
detection (9) of the type of movement;
calculation and memorisation (11) of the corporeal movement, velocity and energy.

2. A procedure according to claim 1, characterised by the fact that the calibration phase (5) is composed of the following phases in succession:
pressing of an activation element (19);
execution of a walk for a set distance;
end of walk.

3. A procedure according to claim 1, characterised by the fact that the calibration phase (5) ends when at least one acceleration sensor (15) stops detecting any movement.

4. A procedure according to claim 3, characterised by the fact that before exiting the calibration phase (5) the microcontroller element (16) updates and saves all the constants of the algorithms rendered operative.

5. A procedure according to claim 1, characterised by the fact that said procedure utilises the data supplied by one or more of the acceleration sensors elements (15) and from said one or more temperature sensor elements (18), all of said sensors being located inside a shoe.

6. A procedure according to claim 1, characterised by the fact that the data detected by at least one acceleration sensor element (15) and at least one temperature sensor (18) are transmittable via the Internet.

7. A device (20) for the acquisition and treatment of data linked to human energy consumption during daily activity and/or sporting practices, of the type constituted of at least one printing circuit (13) bearing, fixed integrally thereto, at lest one acceleration sensor element (15), a voltage generation element (17), an activation element (19) and at least one temperature sensor element (18), characterised by the fact that said device is positioned inside a shoe (12) and is fitted with at least one microcontroller element (16); said microcontroller element being suitable to both convert and process the signals supplied by the at least one acceleration sensor (15).

8. A device according to claim 7, characterised by the fact that the positioning thereof is inside a sole (14), said positioning guaranteeing the reduction of vibrations and of the rotation effect of the foot during the movement.

9. A device according to claim 7, characterised by the fact that the activation element (19) is activatable by pressure on the ground applied by the foot wearing the shoe (12).

10. A device according to claim 7, characterised by the fact that said device is fitted with an interface element.
